(19) United States
(12) Patent Application Publication
(10) Pub. No.: US 2026/0137554 A1
(43) Pub. Date: May 21, 2026

(54) Facial Cold Plunge Device for Positioning in Cold Plunge Tub

(71) Applicant: Nick Reed, Sheridan, WY (US)

(72) Inventor: Nick Reed, Sheridan, WY (US)

(21) Appl. No.: 19/060,755

(22) Filed: Feb. 23, 2025

Related U.S. Application Data
(63) Continuation-in-part of application No. 18/955,946, filed on Nov. 21, 2024.

(51) Int. Cl. A61F 7/00; A61M 16/20

(52) U.S. Cl. CPC A61F 7/0085; A61F 2007/0003; A61F 2007/0063; A61F 2007/0069; A61F 2007/0086; A61M 16/201

(57) ABSTRACT

The present invention provides a novel apparatus for facilitating cold water immersion therapy specifically targeting the face, to be placed in a newly manufactured or preexisting cold plunge tub. The device is configured to draw intake from the cold plunge tub and return water for circulation back to the tub. A retractable breathing apparatus is operably affixed to the device, allowing users to sustain facial submersion while maintaining respiration, thereby enabling extended immersion durations without discomfort or interruption.

### CROSS-REFERENCE TO RELATED APPLICATION

This application is a continuation in part of U.S. patent application Ser. No. 18/955,946 and claims the benefit of priority thereof.

### BACKGROUND OF THE INVENTION

The present invention relates generally to devices and systems for cold therapy, particularly to portable, compact apparatuses designed for facial immersion in cold water. More specifically, this invention pertains to a facial cold plunge device with integrated chilling, filtration, and a retractable breathing apparatus, enabling prolonged and convenient cold water immersion for therapeutic, cosmetic, and wellness applications. The present invention is designed to be placed in a newly manufactured or preexisting cold plunge tub.

### BRIEF SUMMARY OF THE INVENTION

The present invention provides a novel apparatus for facilitating cold water immersion therapy specifically targeting the face, to be placed in a newly manufactured or preexisting cold plunge tub. The device is configured to draw intake from the cold plunge tub and return water for circulation back to the tub. A retractable breathing apparatus is operably affixed to the device, allowing users to sustain facial submersion while maintaining respiration, thereby enabling extended immersion durations without discomfort or interruption.

Thie invention allows for easy integration with existing cold plunge tubs and addresses shortcomings of existing cold therapy methods by eliminating the need for external ice, frequent water changes, and manual temperature adjustments, while providing a safer, more convenient, and effective method for engaging in cold therapy.

The invention is particularly applicable in health and wellness industries, providing benefits such as stress reduction, vagus nerve stimulation, inflammation reduction, and improved skin tone and texture. The invention also offers a cost-effective adjunct to full-body cold plunges and is far superior to DIY facial immersion solutions.

### BRIEF DESCRIPTION OF THE FIGURES

FIG. 1: illustrates the key components of the facial cold plunge device configured to operate in conjunction with an existing cold plunge tub.

### DETAILED DESCRIPTION

The present invention is directed to a novel and advanced facial cold plunge device, for use in conjunction with a cold plunge tub. The invention comprises a main housing unit fabricated from thermally insulated materials, such as high-grade thermoplastics or stainless steel, designed to maintain the thermal integrity of the internal water reservoir.

The housing contains an ergonomically contoured, bowl-shaped reservoir specifically designed to conform to the user's facial profile, enhancing comfort and immersion efficiency while minimizing water spillage. Embedded beneath the reservoir may be optionally an integrated chilling mechanism employing a miniature compressor or thermoelectric cooling system. This system can incorporate evaporative cooling coils positioned strategically to ensure uniform cooling across the reservoir, maintaining water temperatures below 50° F.

The chilling mechanism can be controlled via an electronic thermostat interface, equipped with a digital display for real-time temperature readings and programmable settings, allowing the user to customize and maintain precise water temperatures for optimal therapeutic effect.

The tube is mechanically retractable via a spring-based or tensioned arm mechanism, allowing it to be stowed when not in use. The breathing tube is further equipped with a two-way check valve system that regulates airflow, preventing water ingress while facilitating effortless inhalation and exhalation during submersion. The height and position of the breathing tube can be adjusted to align with the user's facial orientation, enhancing comfort and usability.

The present is designed to attach to the wall of a full-body cold plunge, enabling the integration of facial immersion therapy into an existing system. In this configuration, the device features an adaptable mounting mechanism that securely affixes the ergonomic reservoir to the rim or sidewall of a full-body plunge tub.

A submersible pump, operatively connected to the main unit, draws water from the full-body plunge tub into the facial immersion bowl and recirculates it back into the tub. The device may further incorporate a filtration unit. The pump continuously circulates water through a filtration assembly, which includes replaceable carbon or HEPA filters to remove impurities, bacteria, and debris, ensuring the water remains clean and suitable for repeated use without requiring frequent draining or replacement.

A unique feature of the invention is the retractable breathing apparatus, which allows users to sustain facial submersion for extended durations without the need to interrupt the immersion process to breathe. This apparatus comprises a snorkel-like breathing tube constructed of medical-grade silicone, which is mounted on the housing unit adjacent to the reservoir.

The breathing apparatus, ergonomic reservoir, and optional UV sterilization components ensure users experience the same therapeutic and cosmetic benefits while leveraging existing equipment.

This configuration enhances the versatility and cost-effectiveness of the invention, making it accessible to users who already utilize full-body cold plunge systems but desire the convenience and specificity of facial cold therapy.

### DETAILED DESCRIPTION OF FIGURES

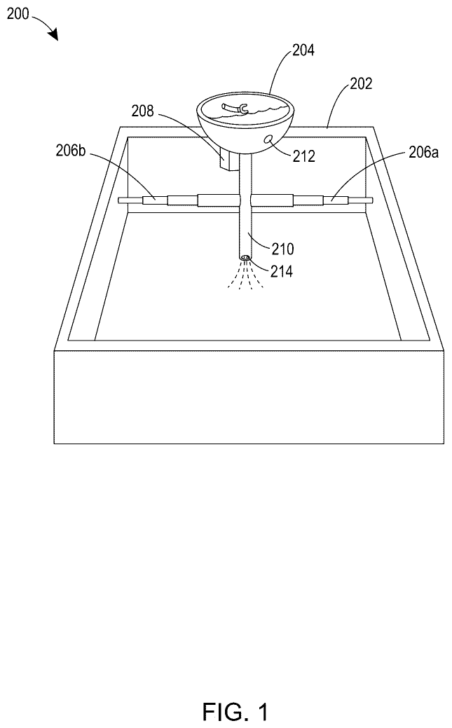

FIG. 1: illustrates the structural and functional components of the facial cold plunge device used in conjunction with a cold plunge tub. The following components are labeled with their respective reference numerals:

### 200: Integrated Assembly of Cold Plunge Tub and Facial Plunge Device

In this schematic, the facial cold plunge device sits in a cold plunge tub which contains its own water reservoir.

### 202: Cold Plunge Tub

The cold plunge tub can be purpose built to operate with the facial cold plunge or the facial cold plunge can be retrofitted into an existing cold plunge tub.

### 204: Facial Cold Plunge Lowering Attached Breathing Mechanism

The attached breathing mechanism is a retractable snorkel-like tube that lowers into position for use. This component includes an adjustable height mechanism and can contain a two-way check valve to allow seamless respiration while preventing water ingress during facial immersion.

### 206a and 206b: Telescoping Legs

The telescoping legs are configured to make the facial cold plunge device adjustable for a variety of cold plunge tub designs as well as adjustable. They can extend and retract to position the facial cold plunge suitably for operation by a user sitting in the cold plunge tub.

### 208: Pump

A submersible pump, operatively connected to the main unit, draws water from the full-body plunge tub into the facial immersion bowl and recirculates it back into the tub. This closed-loop circulation system eliminates the need for separate chilling and filtration mechanisms within the facial device itself, as the water is maintained at the desired temperature and cleanliness by the full-body plunge's primary systems.

### 210: Inlet Pipe

The inlet pipe carries water from the cold plunge tub reservoir. The circulation system may also incorporate insulated conduits to prevent thermal losses during operation, ensuring consistent and efficient cooling.

### 212: Outlet Opening

Water from the facial cold plunge is exhausted through the outlet opening. In some embodiments, there may be a conduit from the outlet opening to either an external drain for the water to exit the system or to a filtration unit with the cold plunge tub for filtration before return to the cold plunge tub reservoir.

### 214: Inlet Opening

Water from the cold plunge tub reservoir enters through the inlet opening and flows up the inlet pipe due to the suction force provided by the pump.

What is claimed is:

1) A facial cold plunge device, comprising:
a) a housing unit fabricated from thermally insulated material;
b) an ergonomically contoured, bowl-shaped facial cold plunge reservoir integrated into the housing unit and configured to accommodate a user's face for water immersion;
c) an inlet opening from a cold plunge tub reservoir to an inlet pipe;
d) an outlet opening;
e) a water circulation system, comprising:
i) a pump configured to circulate water from the cold plunge tub reservoir to the facial cold plunge reservoir; and
ii) insulated conduits connecting the pump to the reservoir;
f) a retractable breathing apparatus mounted to the housing unit, the breathing apparatus comprising:
i) a snorkel-like breathing tube constructed from medical-grade silicone;
ii) a two-way check valve for regulating airflow and preventing water ingress; and
iii) an adjustable positioning mechanism for aligning the breathing tube with a user's facial orientation during water immersion.

2. The facial cold plunge device of claim 1, wherein the reservoir is contoured to provide a watertight seal against the user's face to minimize water spillage during use.

3. The facial cold plunge device of claim 1, wherein the chilling mechanism is operable to cool water within the reservoir to temperatures as low as 32° F. while maintaining uniform cooling across the reservoir surface.

4. The facial cold plunge device of claim 1, wherein the filtration assembly comprises a carbon or HEPA filter capable of removing bacteria, debris, and other impurities from the water during operation.

5. The facial cold plunge device of claim 1, wherein the retractable breathing apparatus is operatively mounted on a spring-loaded mechanism to allow stowing when not in use and deployment during immersion.

6. The facial cold plunge device of claim 1, wherein the two-way check valve of the breathing apparatus is configured to allow seamless inhalation and exhalation while submerged in water.

7. The facial cold plunge device of claim 1, wherein the thermostat control system includes a programmable timer function to automatically maintain the desired water temperature over a predefined period.

8. The facial cold plunge device of claim 1, wherein the housing unit comprises a compact, lightweight design with rubberized feet or suction cups for enhanced stability during operation.

9. The facial cold plunge device of claim 1, further comprising an optional battery-powered mode to enable portable and off-grid use.

10. The facial cold plunge device of claim 1, wherein the water circulation system is configured to operate continuously to maintain consistent water temperature and filtration during extended periods of use.

11. The facial cold plunge device of claim 1, wherein the breathing apparatus is adjustable in length and height to accommodate users of varying facial dimensions and preferences.

12. The facial cold plunge device of claim 1, wherein the ergonomic reservoir is constructed with non-slip interior coatings to enhance user safety and comfort during immersion.

13. The facial cold plunge device of claim 1, wherein the digital display of the thermostat system provides real-time feedback on water temperature and system status.

14. The facial cold plunge device of claim 1, further comprising an integrated safety mechanism configured to shut down the chilling mechanism and pump in the event of water leakage or overheating.

15. The facial cold plunge device of claim 1, wherein the device is configured to support detachable accessories, including alternative filtration systems and enhanced breathing apparatus configurations.

16. The facial cold plunge device of claim 1, wherein the device is configured to attach to the wall of a full-body cold plunge system and further comprising:
a) a mounting mechanism adapted to secure the reservoir to the rim or sidewall of the full-body cold plunge tub;
b) a submersible pump operatively connected to the reservoir, configured to draw water from the full-body plunge tub into the reservoir and return it, thereby utilizing the primary chilling and filtration systems of the full-body plunge; and
c) a closed-loop circulation system enabling continuous water flow between the full-body cold plunge tub and the reservoir, while maintaining ergonomic and respiratory functionalities of the facial cold plunge device.

17. The facial cold plunge device of claim 16, wherein the mounting mechanism comprises two or more telescoping legs.

18. The facial cold plunge device of claim 1, further comprising a filtration mechanism disposed within the facial cold plunge housing.
